(19) United States
(12) Patent Application Publication
(10) Pub. No.: US 2012/0270900 A1
(43) Pub. Date: Oct. 25, 2012

(54) NOVEL METHOD OF TREATMENT

(75) Inventors: Uffe Olesen, Kobenhavn (DK); Annemette Thougaard, Kobenhavn (DK); Maxwell Sehested, kobenhavn (DK)

(73) Assignee: Topo Target A/S, Kobenhavn (DK)

(21) Appl. No.: 13/384,559

(22) PCT Filed: Jul. 16, 2010
(86) PCT No.: PCT/EP10/60302

§ 371 Date: Jul. 12, 2012

Related U.S. Application Data
(60) Provisional application No. 61/226,465, filed on Jul. 17, 2009.

(51) Int. Cl. A61K 31/4545; A61K 31/455; G01N 33/573; A61P 35/00

(52) U.S. Cl. 514/318; 435/7.4; 436/501

(57) ABSTRACT

The present application discloses a method for the treatment or for alleviating the symptoms of a cancer in a subject comprising the steps of a) determining the level of Nicotinic acid phosphoribosyltransferase (NAPRT) in said subject; and b) 1) in the event of a level of NAPRT which is lower than a predetermined threshold value, treating said subject sequentially/simultaneous with i) an effective amount of a nicotinamide phosphoribosyltransferase inhibitor (NAMPRTi), and ii) an effective amount of a nicotinic acid, a nicotinic acid precursor or a prodrug of nicotinic acid; or 2) in the event of a level of NAPRT which is higher than or equal to a predetermined threshold value, treating said subject with i) an effective amount of a NAMPRTi in the absence of sequential/simultaneous treatment with ii) an effective amount of a nicotinic acid, a nicotinic acid precursor or a prodrug of nicotinic acid.

### FIELD OF THE INVENTION

The present invention relates to biomarkers useful in a method for predicting the utility of administering a vitamin PP compound to reduce the severity of side-effects of cancer treatment with therapeutic agents such as inhibitors of the enzyme nicotinamide phosphoribosyltransferase (NAMPRT).

### BACKGROUND OF THE INVENTION

Inhibition of the enzyme nicotinamide phosphoribosyltransferase (NAMPRT) results in the inhibition of NF-kB, the inhibition of NF-kB being a result of the lowering of cellular concentrations of nicotinamide adenine dinucleotide (NAD) (Beauparlant et al. (2007) AACR-NCI-EORTC International Conference on Molecular Targets and Cancer Therapeutics, 2007 Oct. 22-26 Abstract nr A82; and Roulson et al. (2007) AACR-NCI-EORTC International Conference on Molecular Targets and Cancer Therapeutics, 2007 Oct. 22-26 Abstract nr A81). Tumour cells have elevated expression of NAMPRT and a high rate of NAD turnover due to high ADP-ribosylation activity required for DNA repair, genome stability, and telomere maintenance making them more susceptible to NAMPRT inhibition than normal cells. This also provides a rationale for the use of compounds of this invention in combination with DNA damaging agents for future clinical trials.

The pathways of NAD biosynthesis are shown in FIG. 1.

NAMPRT is involved in the biosynthesis of nicotinamide adenine dinucleotide (NAD) and NAD(P). NAD can be synthesized in mammalian cells by three different pathways starting either from tryptophan via quinolinic acid, from nicotinic acid (niacin) or from nicotinamide (niacinamide).

Quinolinic acid reacts with phosphoribosyl pyrophosphate to form niacin mononucletide (dNAM) using the enzyme quinolinic acid phosphoribosyltransferase [symbol] which is found in liver kidney and brain.

Nicotinic acid (niacin) reacts with PRPP to form niacin mononucleotide (dNAM), using the enzyme niacin phosphoribosyltransferase [symbol] which is widely distributed in various tissues.

Nicotinamide (niacinamide) reacts with PRPP to give niacinamide mononucleotide (NAM) using the enzyme nicotinamide phosphoribosyltransferase (NAMPRT) [symbol] which is also widely distributed in various tissues.

The subsequent addition of adenosine monophosphate to the mononucleotides results in the formation of the corresponding dinucleotides: Niacin mononucleotide and niacinamide mononucleotide react with ATP to form niacin adenine dinucleotide (dNAD) and niacinamide adenine dinucleotide (NAD) respectively. Both reactions, although they take place on different pathways, are catalysed by the same enzyme, NAD pyrophosphorylase [symbol] .

A further amidation step is required to convert niacin adenine dinucleotide (dNAD) to niacinamide adenine dinucleotide (NAD) The enzyme which catalyses this reaction is NAD synthetase [symbol] . NAD is the immediate precursor of niacinamide adenine dinucleotide phosphate (NAD(P)) The reaction is catalysed by NAD kinase. For details see, e.g., Cory J. G. Purine and pyrimidine nucleotide metabolism In: Textbook of Biochemistry and Clinical Correlations 3^{rd} edition ed. Devlin, T, Wiley, Brisbane 1992, pp 529-574.

Normal cells can typically utilize both precursors niacin and niacinamide for NAD(P) synthesis, and in many cases additionally tryptophan or its metabolites. Accordingly, murine glial cells use niacin, niacinamide and quinolinic acid (Grant et al. (1998) J. Neurochem. 70: 1759-1763). Human lymphocytes use niacin and niacinamide (Carson et al. (1987) J. Immunol. 138: 1904-1907; Berger et al. (1982) Exp. Cell Res. 137; 79-88). Rat liver cells use niacin, niacinamide and tryptophan (Yamada et al. (1983) Int. J. Vit. Nutr. Res. 53: 184-1291; Shin et al. (1995) Int. J. Vit. Nutr. Res. 65: 143-146; Dietrich (1971) Methods Enzymol. 18B; 144-149). Human erythrocytes use niacin and niacinamide (Rocchigiani et al. (1991) Purine and pyrimidine metabolism in man VII Part B ed. Harkness et al. Plenum Press New York pp 337-3490). Leukocytes of guinea pigs use niacin (Flechner et al. (1970), Life Science 9: 153-162).

NAD(P) is involved in a variety of biochemical reactions which are vital to the cell and have therefore been thoroughly investigated. The role of NAD(P) in the development and growth of tumours has also been studied. It has been found that many tumour cells utilize niacinamide for cellular NAD(P) synthesis. It is thought that niacin and tryptophan which constitute alternative precursors in many normal cell types cannot be utilized in tumour cells, or at least not to an extent sufficient for cell survival. Selective inhibition of an enzyme which is only on the niacinamide pathway (such as NAMPRT) would constitute a method for the selection of tumour specific drugs. This is exemplified by the NAMPRT inhibitors which have been in clinical trials as anti cancer agents, namely FK866/APO866, (see Hasmann and Schemainda, Cancer Res 63(21):7463-7442.), CHS828/GMX1778 and its prodrug EB1627/GMX1777 (see Hjarnaa et al, Cancer Research 59; 5751-5757; Binderup et al, Bioorg Med Chem Lett 15:2491-2494). Further inhibitors of NAMPRT are found in WO 2006/066584, WO 2003/097602, WO 2003/097601, WO 2002/094813, WO 2002/094265, WO 2002/042265, WO 2000/61561, WO2000/61559, WO 1997/048695, WO 1997/048696, WO 1997/048397, WO 1999/031063, WO 1999/031060 and WO 1999/031087.

The administration of NAMPRT inhibitors is associated with gastrointestinal toxicity and myelosuppression (Ravaud et al. Eur J. Cancer 41:702-707; Hovstadius et al. Clin. Cancer Res. 8:2843-2850; WO 1999/053920). This toxicity has been circumvented to some extent by using sub-optimal doses of the NAMPRTi, use of a prodrug and by switching from oral to i.v. administration (Binderup et al. Bioorg Med Chem Lett 15:2491-2494). This toxicity can be substantially alleviated by vitamin PP compounds, which neutralise the cytotoxic effect of the NAMPRTi APO866 on primary lymphocytes and primary intestinal cells. Unfortunately it was observed that the vitamin PP compounds also neutralise the cytotoxicity of the NAMPRTi APO866 on leukemic cells (see WO 1999/053920) and the vitamin PP compound nicotinic acid abrogates the antitumour effect of the NAMPRTi GMX1777 on myeloma unless the nicotinic acid is given 24 hours after the administration of the NAMPRTi (Beauparlant et al. Anti-cancer drugs 20[5]: 346-354.) Beauparlant et al. suggest that nicotinic acid could be useful in case of accidental overdose of an NAMPRTi.

The prior art has not been consistent in the use of abbreviations for the enzymes in NAD metabolism. For the avoidance of doubt the instant specification deals with the following enzymes:

| | Enzyme | |
|---|---|---|
| Name | classification | Abbreviation |
| Nicotinamide phosphoribosyl transferase | EC 2.4.2.12 | NAMPRT |
| Nicotinic acid phosphoribosyltransferase | EC 2.4.2.11 | NAPRT |

### SUMMARY OF THE INVENTION

The present invention demonstrates that NAPRT expression in a target cell, such as a tumour cell, acts as a marker for protection against NAMPRT inhibitors by vitamin PP compounds such as nicotinic acid, a nicotinic acid precursor or a prodrug of nicotinic acid, such as nicotinic acid ester. This discovery has opened up a new avenue for the stratification of subjects prior to or during treatment with NAMPRT inhibitors. Selected vitamin PP compounds such as nicotinic acid, nicotinic acid precursors or prodrugs of nicotinic acid, and related compounds can be used to alleviate the toxic side effects of NAMPRT inhibitors, maintaining anti-tumour activity of the NAMPRT inhibitors; the therapeutic window is widest when tumours have the lowest expression of NAPRT.

Hence, it has been found by the present inventor(s) that it is beneficial to sequentially or simultaneously administer an effective amount of a NAMPRT inhibitor and a nicotinic acid, a nicotinic acid precursor or a prodrug of nicotinic acid if the tumours have a low expression of NAPRT.

So, in a first aspect the present invention relates to a method for the treatment or for alleviating the symptoms of a cancer in a subject, the method comprising the steps of a) determining the level of Nicotinic acid phosphoribosyltransferase (NAPRT) in said subject; and b) 1) in the event of a level of NAPRT, as determined in step a) above, which is lower than a predetermined threshold value, treating said subject sequentially or simultaneous with i) an effective amount of a nicotinamide phosphoribosyltransferase inhibitor (NAMPRTi), and ii) an effective amount of a nicotinic acid, a nicotinic acid precursor or a prodrug of nicotinic acid; or 2) in the event of a level of NAPRT, as determined in step a) above, which is higher than or equal to a predetermined threshold value, treating said subject with i) an effective amount of a nicotinamide phosphoribosyltransferase inhibitor (NAMPRTi) in the absence of sequential or simultaneous treatment with ii) an effective amount of a nicotinic acid, a nicotinic acid precursor or a prodrug of nicotinic acid.

The present invention also relates to the use of Nicotinic acid phosphoribosyltransferase (NAPRT) as a biomarker in selecting responsive patients to the sequential or simultaneous treatment with i) an effective amount of a nicotinamide phosphoribosyltransferase inhibitor (NAMPRTi), and ii) an effective amount of a nicotinic acid, a nicotinic acid precursor or a prodrug of nicotinic acid; and to the use of Nicotinic acid phosphoribosyltransferase (NAPRT) as a biomarker in selecting patients that benefit from being treated with an effective amount of a nicotinamide phosphoribosyltransferase inhibitor (NAMPRTi) in the absence of sequential or simultaneous treatment with an effective amount of a nicotinic acid, a nicotinic acid precursor or a prodrug of nicotinic acid.

Further, the present invention relates to the use of a nicotinamide phosphoribosyltransferase inhibitor (NAMPRTi) in the preparation of a medicament for the treatment or for alleviating the symptoms of a cancer in a subject, the treatment comprising the steps of a) determining the level of Nicotinic acid phosphoribosyltransferase (NAPRT) in said subject; and b)1) in the event of a level of NAPRT, as determined under step a) above, which is lower than a predetermined threshold value, treating said subject sequentially or simultaneous with i) an effective amount of a nicotinamide phosphoribosyltransferase inhibitor (NAMPRTi), and ii) an effective amount of a nicotinic acid, a nicotinic acid precursor or a prodrug of nicotinic acid; or 2) in the event of a level of NAPRT, as determined under step a) above, which is higher than or equal to a predetermined threshold value, treating said subject with i) an effective amount of a nicotinamide phosphoribosyltransferase inhibitor (NAMPRTi) in the absence of sequential or simultaneous treatment with an effective amount of a nicotinic acid, a nicotinic acid precursor or a prodrug of nicotinic acid.

In a further aspect the present invention relates to a method for alleviating the side effects of a nicotinamide phosphoribosyltransferase inhibitor (NAMPRTi) in the treatment with an effective amount of said NAMPRTi of a cancer in a subject, the method comprising the steps of a) determining the level of Nicotinic acid phosphoribosyltransferase (NAPRT) in said subject; and b) in the event of a level of NAPRT, as determined in step a) above, which is lower than a predetermined threshold value, treating said subject with an effective amount of a nicotinic acid, a nicotinic acid precursor or a prodrug of nicotinic acid, sequentially or simultaneous with the treatment with said effective amount of a nicotinamide phosphoribosyltransferase inhibitor (NAMPRTi). In some embodiments the side effects are in normal tissue, such as lymphocytes and primary intestinal cells.

### BRIEF DESCRIPTION OF THE FIGURES

FIG. 1 illustrates the pathway of NAD synthesis (from Biedermann E et al, WO 00/50399).

FIG. 2 illustrates the cumulative survival of mice in response to high dose APO866 treatment. Treatment is 60 mg APO866 twice/day for 4 days. NA=nicotinic acid.

FIG. 3 illustrates the tail vein platelet counts on the last treatment day in mice treated with APO866 40 mg/kg i.p. ×2/day for 4 days, ±nicotinic acid (NA) 20 mg/kg ×1/day p.o. for five days (NA treatment started on the day before APO866 treatment). A vehicle control group is included for comparison. The result of a t-test is shown on the figure.

FIG. 4 illustrates the cumulative survival of mice with subcutaneous A2780 xenografts: Time used for each individual mouse's tumour to reach a size of 800 mm3. The mice were treated i.p. with doses of 15 or 50 mg/kg APO866×2/day in two weekly 4-day cycles combined with vehicle p.o. or 50 mg/kg nicotinic acid (NA). Legend on the figure: The p-values of log-rank analysis comparing the individual groups are shown on the figure.

FIG. 5 illustrates the cumulative survival of mice with subcutaneous ML-2 xenografts: Time used for each individual mouse's tumour to reach a size of 800 mm3. The mice were treated i.p. with doses of 15 or 50 mg/kg APO866×2/day in two weekly 4-day cycles combined with vehicle p.o. or 50 mg/kg nicotinic acid (NA). Legend on the figure: The p-values of log-rank analysis comparing the individual groups are shown on the figure.

FIG. 6 illustrates the expression of NAPRT mRNA relative to actin in different cancer cell lines.

FIG. 7 illustrates cell viability in the ovarian cancer cell line A2780 measured by CellTiterGlo® after 3 days of CHS-828 treatment with and without 1 mM nicotinic acid added to the medium.

FIG. 8 illustrates cell viability in the colon cancer cell line HCT116 measured by CellTiterGlo® after 3 days of compound 1050 treatment with and without varying concentrations of nicotinic acid added to the medium.

FIG. 9 illustrates cell viability in the small cell lung cancer cell line NYH measured by CellTiterGlo® after 3 days of compound 1050 treatment with and without 1 mM nicotinic acid added to the medium.

FIG. 10 illustrates the protein levels of NAPRT in cell lines protected by nicotinic acid (ML-2, HCT-116 and A431; 1, 2 and 3, respectively) and in cells not protected by nicotinic acid (A2780, NYH and PC-3; 4, 5 and 6, respectively).

FIG. 11 illustrates cells protected and unprotected against NAMPRT inhibitors by nicotinic acid; no positive reactivity for NAPRT in PC-3 (FIG. 11 A+C); strong reactivity for NAPRT in HCT-116 cells (FIG. 11 B+D).

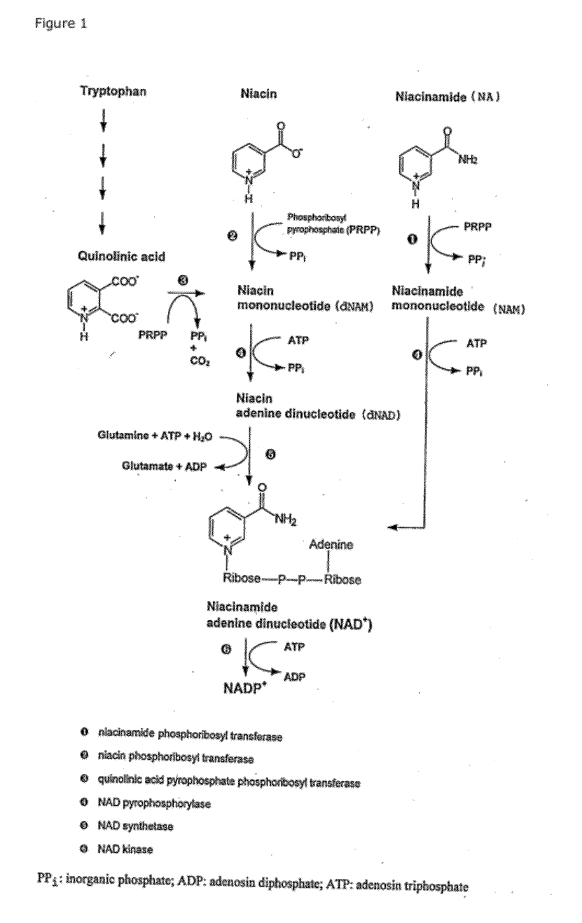

### DETAILED DISCLOSURE OF THE INVENTION

### Method of the Invention

As mentioned above, the present invention i.a. relates to a method for the treatment or for alleviating the symptoms of a cancer in a subject, the method comprising the steps of

a) determining the level of Nicotinic acid phosphoribosyltransferase (NAPRT) in said subject; and
b) 1) in the event of a level of NAPRT, as determined in step a) above, which is lower than a predetermined threshold value, treating said subject sequentially or simultaneous with i) an effective amount of a nicotinamide phosphoribosyltransferase inhibitor (NAMPRTi), and ii) an effective amount of a nicotinic acid, a nicotinic acid precursor or a prodrug of nicotinic acid; or
2) in the event of a level of NAPRT, as determined in step a) above, which is higher than or equal to a predetermined threshold value, treating said subject with i) an effective amount of a nicotinamide phosphoribosyltransferase inhibitor (NAMPRTi) in the absence of sequential or simultaneous treatment with ii) an effective amount of a nicotinic acid, a nicotinic acid precursor or a prodrug of nicotinic acid.

Step a)

A key step of the method of the invention is that of determining the level of nicotinic acid phosphoribosyltransferase (NAPRT) in the subject in question. The present findings allow the stratification and/or selection of subjects for either 1) the combined treatment with an inhibitor of NAMPRT (NAMPRTi) and a nicotinic acid, a nicotinic acid precursor or a prodrug of nicotinic acid, in particular nicotinic acid or a prodrug thereof, or 2) the treatment of treatment with an inhibitor of NAMPRT (NAMPRTi) in the absence of a nicotinic acid, a nicotinic acid precursor or a prodrug of nicotinic acid.

The stratification of the subjects is based on a predetermined threshold value which, e.g., is set by the medical practitioner based data from a plurality of patients, e.g. at least 5 patient, or at least 20 patient, or even at least 50 patients.

Hence, in order to create basis for setting the threshold value, it will be necessary to first establish or obtain data from a cohort of existing patients to determine the level of NAPRT in their tumour tissue. The level of NAPRT in tumour tissue may be determined by one of a number of methods which either directly measure NAPRT, or which in a more indirect manner correlates (or is expected to correlate) with the level of NAPRT in the tissue in question.

The cohort to which reference is made is desirably matched to one or more of tumour type, age, sex, or severity of disease, in particular the tumour type.

In one variant, however, the threshold value may set based on the level of NAPRT of a different tissue type than the tumour tissue in a population of human beings. This may be similar or identical patients, or may alternatively be healthy subjects. However, preferably, the threshold value is set based on the level of NAPRT in the same tissue, such as tumour tissue, as the tumour tissue in question, and obtained from plurality of patients with the same cancer indication.

The level of NAPRT in the tissue in question (of the subject in question) and for the purpose of setting the threshold value may be determined at the level of mRNA expression, e.g. using RT-PCR. In another variant, the level of NAPRT is determined more directly, e.g. using an antibody based approach. Furthermore, the level of NAPRT may be determined on the basis of functional enzyme activity. Any diminution of NAPRT activity in the tumour cell may be caused by low amounts of enzyme, inactive mutants or splice variants of the enzyme, which can be detected by sequencing. Such methods are known per se in the art. In further variants, the level of NAPRT may be determined by means of determining the level of either or both of niacin mononucleotide (dNAM) and niacin adenine dinucleotide (dNAD), the level of which in the tumour tissue be expected to correlate with the level of NAPRT.

In some variants, the level of nicotinic acid phosphoribosyltransferase (NAPRT) is determined on the level of nucleic acids encoding NAPRT, such as by RT-PCR.

In other variants, the level of nicotinic acid phosphoribosyltransferase (NAPRT) is determined on the level of proteins, such as in assays based on specific antibodies or other specific binding partners to NAPRT.

It is to be understood that the level of NAPRT may be determined directly or indirectly from the tumour tissue or tumour cells of the subject. The amount of tumour tissue or cells necessary to determine a correct level of NAPRT may vary from small to larger samples of the tumour or tumour cells, or alternatively the entire tumour and will be dependent on the specific assay used and its sensitivity, all of which is well known to the person skilled in the art. In some embodiments the level of NAPRT is determined from a biological sample within or near the tumour or tumour cells and/or from a biological sample otherwise being indicative of the level of NAPRT in the tumour tissue or tumour cells, such as blood, serum, urine, hair, saliva, skin, tissue, plasma, cerebrospinal fluid (CSF), amniotic fluid, nipple aspirate, sputum, feces, synovial fluid, nails, or the like depending on the specific tumour or tumour cells of the subject.

The expression levels are typically be distributed amongst low, intermediate and high values. It will be appreciated that what is determined to be of a low, intermediate or high value will be to some extent an arbitrary designation depending upon the criteria applied by any one particular treatment centre, in a similar manner to, for example, biochemical markers used in prenatal diagnoses. However this does not prevent the method being practised to the extent that the threshold level of NAPRT can be determined in new subjects and compared to the collected data to establish predictions or dosages in accordance with the present invention.

In most embodiments of the method of the invention, the step of determining the level of NAPRT is followed by the step of the comparing said level in the subject of interest to the threshold level previously set based on the values determined in a cohort of patients.

It will be understood that the step of comparing may be performed on historic data, and that it is not necessary to repeat the determination for that cohort each time the above method is practised.

In some practical embodiments, the predetermined threshold value is set such that values lower than the threshold value are represented by subjects in the lower one-third, preferably the lower quartile, of the distribution of the cohort.

Step b)

In the second step of the method of the invention, the level of NAPRT in the subject of interest is compared to the predetermined threshold value. This comparison provides basis for deciding whether it is beneficial to utilise a nicotinic acid, a nicotinic acid precursor or a prodrug of nicotinic acid (e.g. nicotinic acid) to alleviate the severity of side effects of NAMPRTi treatment (i.e. if the level is lower than the threshold value), or whether it is beneficial to administer, preferably in lower initial doses, the NAMPRT inhibitor in the absence of a nicotinic acid, a nicotinic acid precursor or a prodrug of nicotinic acid (e.g. nicotinic acid).

In the latter instance, it is possible to monitor the side-effects of the therapy, and possible to use a nicotinic acid, a nicotinic acid precursor or a prodrug of nicotinic acid (e.g. nicotinic acid) 24 hours or more after administration of the NAMPRTi to alleviate side effects.

Hence, in the event of a level of NAPRT, as determined in step a) above, which is lower than a predetermined threshold value, treating said subject sequentially or simultaneous with i) an effective amount of a nicotinamide phosphoribosyltransferase inhibitor (NAMPRTi), and ii) an effective amount of a nicotinic acid, a nicotinic acid precursor or a prodrug of nicotinic acid.

Similarly, in the event of a level of NAPRT, as determined in step a) above, which is higher than or equal to a predetermined threshold value, treating said subject with i) an effective amount of a nicotinamide phosphoribosyltransferase inhibitor (NAMPRTi) in the absence of sequential or simultaneous treatment with ii) an effective amount of a nicotinic acid, a nicotinic acid precursor or a prodrug of nicotinic acid.

In one embodiment, the absence of sequential or simultaneous treatment with an effective amount of a nicotinic acid, a nicotinic acid precursor or a prodrug of nicotinic acid under step b)2) is sequential and within 24 hours of treatment.

In another embodiment, The method according to any one of the preceding claims, wherein said subject is treated subsequent and after 24 hours of the treatment under step b) with an effective amount of a nicotinic acid, a nicotinic acid precursor or a prodrug of nicotinic acid.

In some variants of the method of treatment, the nicotinamide phosphoribosyltransferase inhibitor (NAMPRTi) is administered prior to said nicotinic acid, nicotinic acid precursor or prodrug of nicotinic acid.

In other variants of the method of treatment, the nicotinamide phosphoribosyltransferase inhibitor (NAMPRTi) is administered concurrently with the administration of said nicotinic acid, nicotinic acid precursor or prodrug of nicotinic acid.

Inhibitors of NAMPRT

NAMPRT inhibitors suitable for use in the treatment of cancer and other diseases are known in the art. Examples of inhibitors of NAMPRT are found in WO 2009/086835, WO 2009/156421, WO 2010/023307, WO 2010/066709, PCT/EP2010/058102, WO 2006/066584, WO 2003/097602, WO 2003/097601, WO 2002/094813, WO 2002/094265, WO 2002/042265, WO 2000/61561, WO2000/61559, WO 1997/048695, WO 1997/048696, WO 1997/048397, WO 1999/031063, WO 1999/031060 and WO 1999/031087.

Especially interesting examples of NAMPRT inhibitors include the following:

| Compound | Structure |
|---|---|
| APO866/FK866 | 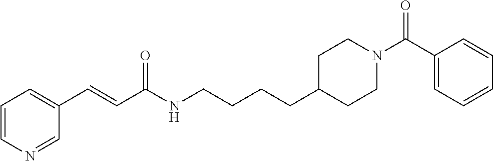 |
| CHS828/GMX1778 | 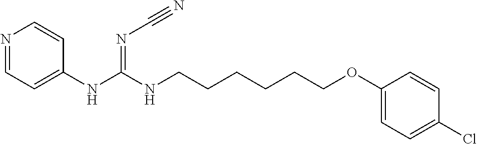 |
| EB1627/GMX1777 | 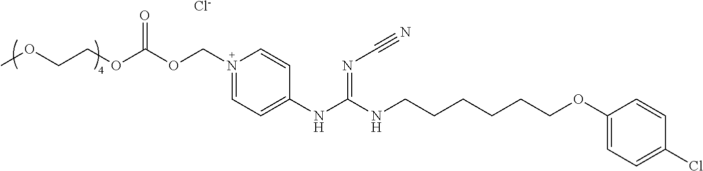 |
| Compound 1050 from WO 2009/086835 | 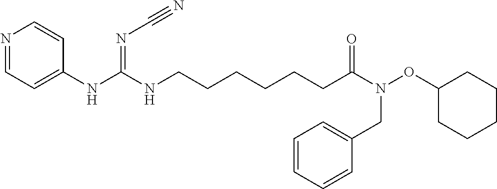 |
| Compound 1077 from WO 2009/156421 | 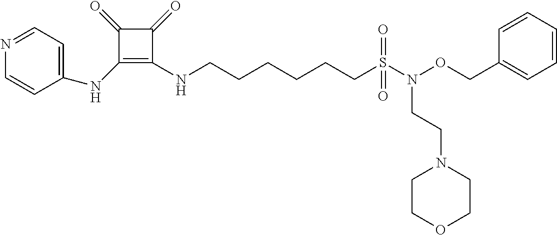 |
| Compound 1082 from WO 2010/023307 | 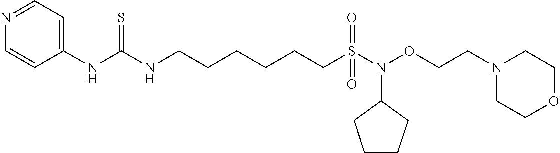 |
| Compound 1001 from WO 2010/066709 | 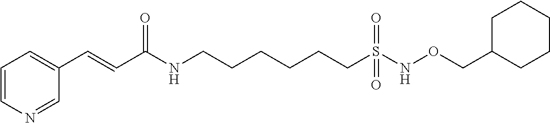 |
| Compound 1010 from PCT/EP2010/58102 | 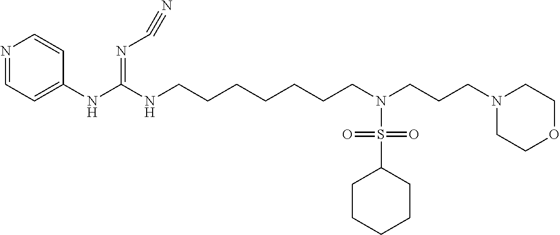 |

All of these compounds or pharmaceutically acceptable salts thereof may be used in accordance with the present invention.

Nicotinic Acid, Nicotinic Acid Precursor and Prodrugs of Nicotinic Acid

The use of Vitamin PP compounds (which encompasses nicotinic acid and derivatives) to alleviate the side effects of NAMPRT inhibitors, is taught in WO 1999/53920. Given the knowledge described in this specification of the key role of NAPRT in the protection of cells from NAMPRT inhibitors, the instant invention appears to be particularly relevant when nicotinic acid, nicotinic acid precursors or prodrugs of nicotinic acid, e.g. nicotinic acid, are used.

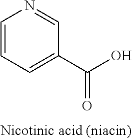

Prodrugs of nicotinic acid are well known in the art. Some examples are shown below.

| Nicotinic acid ester prodrug | CAS No. |
|---|---|
| 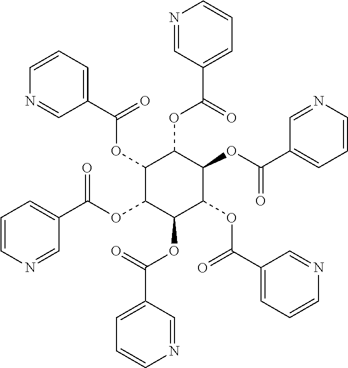 | 6556-11-2 |
| 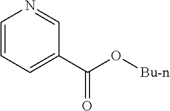 | 6938-06-3 |
| 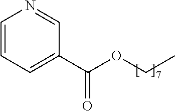 | 70136-02-6 |
| 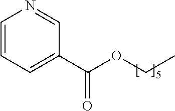 | 23597-82-2 |
| 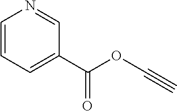 | 24641-06-3 |

In preferred embodiments, the nicotinic acid, a nicotinic acid precursor or a prodrug of nicotinic acid is nicotinic acid.

Treatment of a Tumour

The invention is directed to the treatment of any subject, in particular mammals, such as a human. It should be understood that the method is particularly relevant where the subject (in particular a human) is diagnosed with a cancer, or where the subject is suspected of having a cancer.

In the most typical embodiments, the cancer is selected from cancers of the breast, prostate, lung, colon, cervix, ovary, skin, CNS, bladder, pancreas, leukaemia, and lymphoma.

The method of treatment may further comprise radiation therapy.

Although cancer treatment as described herein requires the use of an anti-cancer agent, preferably an inhibitor of NAMPRT, the treatment may also include additional therapeutic, non-therapeutic or chemotherapeutic agents as described herein.

Reference to a therapeutic regimen comprising the use of an NAMPRT inhibitor includes a regimen consisting of the use of a NAMPRT inhibitor and one or more chemotherapeutic agents, as well as a regimen which comprises the use of an NAMPRT inhibitor, one or more chemotherapeutic agents and one or more additional therapeutic or non-therapeutic agents, as described herein.

In one embodiment, the method of treatment further comprises administering said subject an effective amount of a DNA damaging agent. Examples of DNA damaging agent are for example those selected from Cladribine, Pentostatin, Methotrexate, Trimetrexate glucuronate, Pemetrexed, Treosulfan, Busulfan, Dacarbazine, Temozolomide, Mitomycin C, Chlorambucil, Ifosfamide, Melphalan, Thiotepa, Mechlorethamine, Carmustine, Bendamustin, Fotemustine, Lomustine, Streptozocin, Carboplatin, Cisplatin, Lobaplatin, Oxaliplatin Bleomycin, Hydroxyurea, Actinomycin D, Azacitidine, Decitabine, Nelarabine, Cytarabine, Fludarabine, Clofarabine, Vorinostat, Gemcitabine, 5-Fluorouracil, Capecitabine, Floxuridine, Raltitrexed, Pemetrexed, Irinotecan, Topotecan, Amrubicin, Daunorubicin, Doxorubicin, Epirubicin, Etoposide, Idarubicin, Mitoxantrone, Teniposide, Valrubicin, and Allopurinol, and a pharmaceutically acceptable salt thereof.

As used herein, reference to treatment includes any treatment for the killing or inhibition of growth of a tumour cell. This includes treatment intended to alleviate the severity of a tumour, such as treatment intended to cure the tumour or to provide relief from the symptoms associated with the tumour. It also includes prophylactic treatment directed at preventing or arresting the development of the tumour in an individual at risk from developing a tumour. For example, the treatment may be directed to the killing of micro-metastases before they become too large to detect by conventional means.

In view of the above, the present invention also provides the use of nicotinic acid phosphoribosyltransferase (NAPRT) as a biomarker in selecting responsive patients to the sequential or simultaneous treatment with i) an effective amount of a nicotinamide phosphoribosyltransferase inhibitor (NAMPRTi), and ii) an effective amount of a nicotinic acid, a nicotinic acid precursor or a prodrug of nicotinic acid.

The present invention further provides the use of nicotinic acid phosphoribosyltransferase (NAPRT) as a biomarker in selecting patients that benefit from being treated with an effective amount of a nicotinamide phosphoribosyltransferase inhibitor (NAMPRTi) in the absence of sequential or simultaneous treatment with an effective amount of a nicotinic acid, a nicotinic acid precursor or a prodrug of nicotinic acid.

Still further, the present invention provides the use of a nicotinamide phosphoribosyltransferase inhibitor (NAMPRTi) in the preparation of a medicament for the treatment or for alleviating the symptoms of a cancer in a subject, the treatment comprising the steps of

a) determining the level of Nicotinic acid phosphoribosyltransferase (NAPRT) in said subject;
b) 1) in the event of a level of NAPRT, as measured under step a) above, which is lower than a predetermined threshold value, treating said subject sequentially or simultaneous with i) an effective amount of a nicotinamide phosphoribosyltransferase inhibitor (NAMPRTi), and ii) an effective amount of a nicotinic acid, a nicotinic acid precursor or a prodrug of nicotinic acid; or
2) in the event of a level of NAPRT, as measured under step a) above, which is higher than or equal to a predetermined threshold value, treating said subject with i) an effective amount of a nicotinamide phosphoribosyltransferase inhibitor (NAMPRTi) in the absence of sequential or simultaneous treatment with an effective amount of a nicotinic acid, a nicotinic acid precursor or a prodrug of nicotinic acid.

Dosages

In one embodiment, the pharmaceutical composition is in unit dosage form for each active compound. In such embodiments, each unit dosage form typically comprises 0.1-500 mg, such as 0.1-200 mg, e.g. 0.1-100 mg, of each compound.

More generally, each compound are preferably administered in an amount of about 0.1-250 mg per kg body weight per day, such as about 0.5-100 mg per kg body weight per day.

In some embodiments, the effective amount of the nicotinic acid, nicotinic acid precursor or prodrug of nicotinic acid is administered intravenously at a dose of about 1 mg/day to about 3,000 mg/day, such as in the range of about 10 mg/day to about 1,000 mg/day, such as in the range of about 10 mg/day to about 100 mg/day.

In some variants, the nicotinic acid, nicotinic acid precursor or prodrug of nicotinic acid is administered orally.

For compositions adapted for oral administration for systemic use, the dosage is normally for each compound 0.5 mg to 1 g per dose administered 1-4 times daily for 1 week to 12 months depending on the disease to be treated.

The dosage for each compound for oral administration of the composition in order to prevent diseases or conditions is normally 1 mg to 100 mg per kg body weight per day. The dosage may be administered once or twice daily for a period starting 1 week before the exposure to the disease until 4 weeks after the exposure.

For compositions adapted for rectal use for preventing diseases, a somewhat higher amount of each compound is usually preferred, i.e. from approximately 1 mg to 100 mg per kg body weight per day.

For parenteral administration, a dose for each compound of about 0.1 mg to about 100 mg per kg body weight per day is convenient. For intravenous administration, a dose for each compound of about 0.1 mg to about 20 mg per kg body weight per day administered for 1 day to 3 months is convenient. For intraarticular administration, a dose for each compound of about 0.1 mg to about 50 mg per kg body weight per day is usually preferable. For parenteral administration in general, a solution in an aqueous medium of 0.5-2% or more of each active ingredients may be employed.

For topical administration on the skin, a dose for each compound of about 1 mg to about 5 g administered 1-10 times daily for 1 week to 12 months is usually preferable.

### EXAMPLES

### Materials and Methods

Cell lines: HCT-116, ML-2, A431, PC-3, T24 and A2780 were obtained from ATCC. NYH is previously described—as GLC-2 (Cancer Res. 1985 December; 45(12 Pt 1):6024-33)

Clonogenic assay: Cells were incubated with APO866 at different concentrations with or without 100 μM nicotinic acid and seeded out on semi-solid agar matrix with sheep red blood cells and growth medium. Following a 3-week incubation period, the colonies were counted and % survival relative to control (untreated) cells was calculated. IC₅₀ values were calculated on basis of survival at different concentrations of APO866.

Mouse studies: In toxicity and xenograft studies NMRI mice and nude mice, respectively, were treated once daily p.o. with 0.5% HPMC in water or nicotinic acid in the same vehicle combined with two daily injections of APO866 in PBS/saline with 3% HPβCD. The mice were treated in two weekly four-day cycles.

In xenograft studies, 10⁷ cancer cells were injected s.c. in a mixture of PBS/matrigel in nude athymic mice. The mice were observed daily until tumours started to grow, and treatment with nicotinic acid and APO866, as described above, was initiated when the tumour volumes were around 100 mm³.

mRNA quantification: mRNA from cells was purified using a Trizol (Invitrogen) standard protocol and cDNA was produced by a High Capacity cDNA Archive kit (Applied Biosystems). Expression was analyzed on a 7500 RT-PCR system (Applied Biosystem) using probes for Actin and NAPRT and TaqMan Universal PCR Master Mix Applied Biosystems). The data was analyzed by a method described by Peirson et al. (Nucleic Acids Res. 2003 Jul. 15; 31(14):e73)

CellTiterGlo® luminescent cell viability assay: Cells were plated in opaque 96-well plates (5,000 cells/well) 24 hours before use and then incubated with drug for 72 hours at the indicated concentrations with or without nicotinic acid (Invitrogen) added to the media. The CellTiterGlo® assay (Promega) was performed according to the manufacturer's instructions and bioluminescence was measured. Analysis and determination of IC50 values were performed by Prizm.

Western Blot Analysis

Cells were lysed in a buffer containing 20 mM NaCl, 25 mM MOPS, 2 mM EDTA, 2 mM sodium orthovanadate, 0.1% NP-40, 10% glycerol and 1% Ettan™ protease inhibitor mix (Amersham) using sonication. Protein concentrations were determined by Bio-Rad Protein Assay (Bio-Rad) according to the manufacturer's instructions. Proteins were separated by SDS-PAGE and blotted to a nitrocellulose membrane using the NuPAGE Novex BisTris (XCell SureLock™) system (Invitrogen®). NAPRT primary antibody (ProteinTech Group, #13549-1-AP) was used at a dilution of 1:500 followed by anti-rabbit HRP-conjugated antibody (Amersham) at a dilution of 1:5000. HRP-conjugated GAPDH goat-antibody was purchased from SantaCruz®, and used at a dilution of 1:2000. Detection of HRP-conjugated antibodies was performed with ECL Plus® Blotting Reagent (Amersham).

Immunohistochemical Analysis

Cell pellets were coagulated using plasma and thrombin prior to formalin fixation and paraffin-embedding. For heat-induced epitope retrieval of NAPRT citrate solution (pH 6) was used. A 3% H₂O₂ solution was applied to block endogenous peroxidase activity. The slides were pre-incubated in 1% BSA prior to addition of primary antibodies (incubation: 1 h, room temperature, 1:500). For detection Envision® (DAKO, Glostrup, Denmark, K4011) anti Rabbit and diaminobenzidine/DAB+(DAKO, K3468) was applied. Finally, the slides were counter-stained using Mayer's haematoxylin. To distinguish and visualize reactivity (reddish brown) from no reactivity (blue) in black and white, “Black and white” adjustment in PhotoShop CS3 (Adobe Systems Inc, San Jose, Calif., USA) was applied uniformly using “High Contrast Blue Filter” (preset settings: Reds, Yellows and Greens: −50%, Cyans, Blues and Magentas: 150%) for a darker staining of reddish colors compared with bluish.

Effect of Nicotinic Acid on Maximally Tolerated Dose of APO866 in Mice

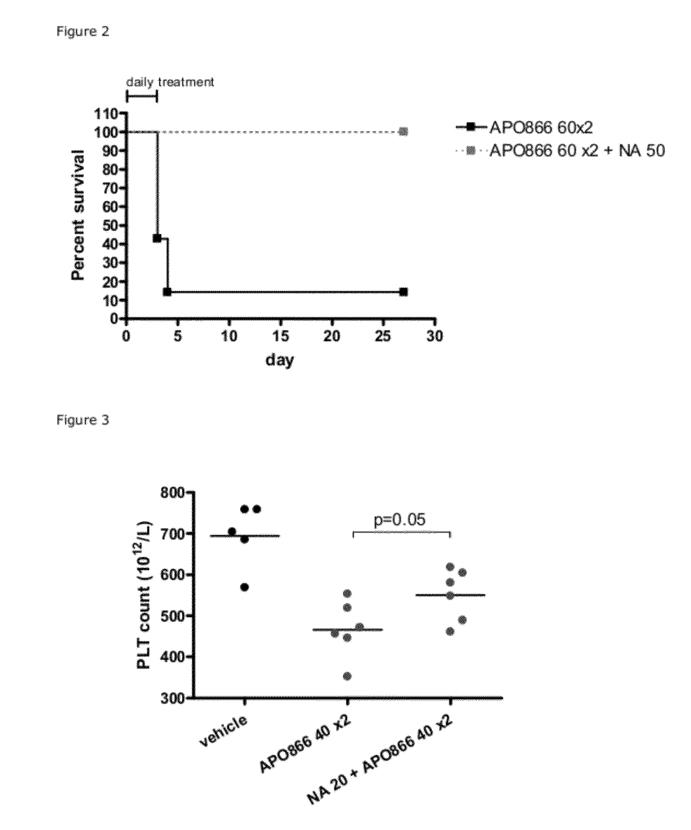

The maximally tolerated dose (MTD) of APO866 in Balb/c nude mice is 15 mg/kg twice a day (data not shown). We examined to which extent dosing nicotinic acid orally could protect mice from APO866-induced death. We treated mice with 60 mg/kg APO866 twice daily on four consecutive days combined with 50 mg/kg/day nicotinic acid p.o., and a control group received only vehicle p.o. As can be seen from FIG. 2, the majority of the control mice died on day 3 and 4. However, if the initial toxicity is survived the mice recover (1 of 7). In comparison, all the mice of the group treated with nicotinic acid survived the APO866 dosing until day 26 where the experiment was terminated. We investigated the effect of the nicotinic acid rescue on blood platelet count (FIG. 3). We found that the platelet count upon APO866 treatment was significantly improved in the group receiving nicotinic acid compared to control.

Protection with Nicotinic Acid Against APO866 In Vitro

Supplement of nicotinic acid to the growth medium can protect against the cytotoxic effects of inhibitors of nicotinamide phosphoribosyltransferase (NAMPRT), including APO866, by synthesizing nicotine adenine dinucleotide (NAD) by an alternative pathway. A cancer cell line, HEPG2, has been shown not to be able to utilize nicotinic acid for NAD synthesis (Cancer Res. 2003 Nov. 1; 63(21):7436-42). We investigated the protective effect of nicotinic acid against APO866 induced cell death in a panel of cell lines of different origin. The cell lines were sensitive to APO866 with IC₅₀ values between 1-13 nM. We used continuous treatment with APO866 in a clonogenic assay. Two cell lines, A2780 and NYH were not protected against APO866 when incubated in a medium containing 100 μM nicotinic acid (Table 1). ML-2, HCT-116 and A431 cells display an increase of IC₅₀ values of more than 40-90 fold in nicotinic acid containing medium compared to standard medium. Addition of 100 μM nicotinic acid to the medium had in itself no effect on the survival of the cells (data not shown)

Protective Effects of Nicotinic Acid on APO866 Treatment of A2780 and ML-2 Xenografts

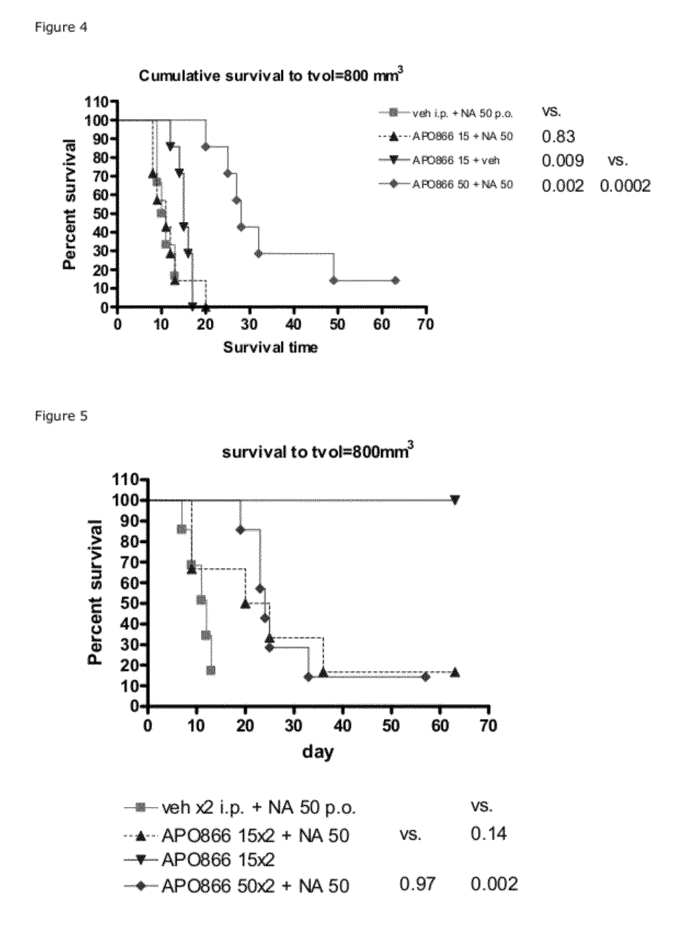

We investigated the in vivo rescue effects of nicotinic acid on nude mice carrying xenograft tumours of A2780 and ML-2 cells. APO866 was given twice daily in weekly four-day cycles for two weeks starting when tumours had reached a size of 100 mm³. Treatment of A2780 xenografts with the MTD dose of 15 mg/kg APO866 i.p. gave an increase of lifespan (% ILS—until tumour size of 800 mm³) of 50% compared to the control group (FIG. 4). In comparison, co-treatment with 50 mg/kg APO866 i.p. and 50 mg/kg nicotinic acid p.o. resulted in an increase in % ILS of 180. Interestingly, although A2780 cells are unable to utilize nicotinic acid we find that 50 mg/kg nicotinic acid negates the anti-proliferative effect of the low dose of 15 mg/kg APO866. The body weight did not vary significantly between the groups during the study (data not shown). Treatment of ML-2 xenografts with APO866 only is very effective with complete elimination of the tumours before day 10 (FIG. 5). If nicotinic acid is co-administered the anti-proliferative effect of the same APO866 dose is partially negated and most tumours persist. The survival in mice given the combination of 15 mg/kg APO866 and nicotinic acid was not significantly different from controls (p=0.14), and increasing the dose of APO866 to 50 mg/kg combined with nicotinic acid does not improve the effect on tumour size in a significant way compared with this group (p=0.97). However, 50 mg/kg treatment with APO866 and nicotinic acid reduced tumour growth when compared with untreated controls (p=0.002). No overall differences in body weight change were seen between the groups of the experiment (data not shown).

Nicotinic Acid Rescue of Diverse NAMPRT Inhibitors in Cancer Cells

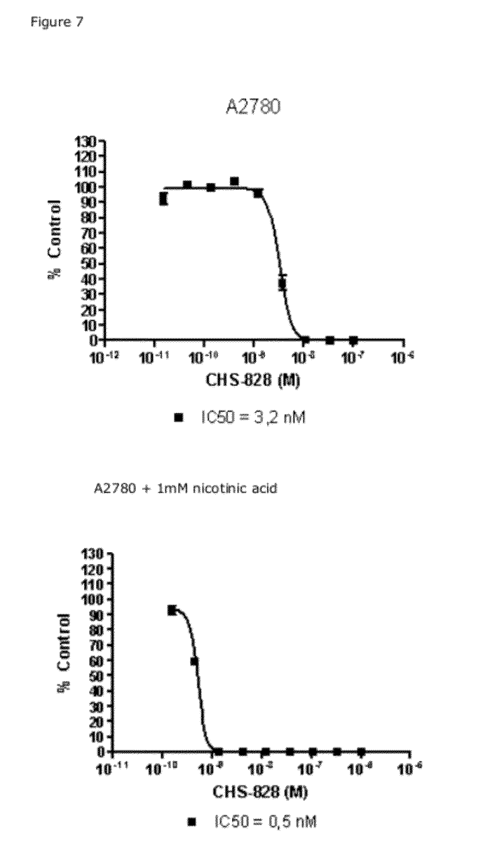

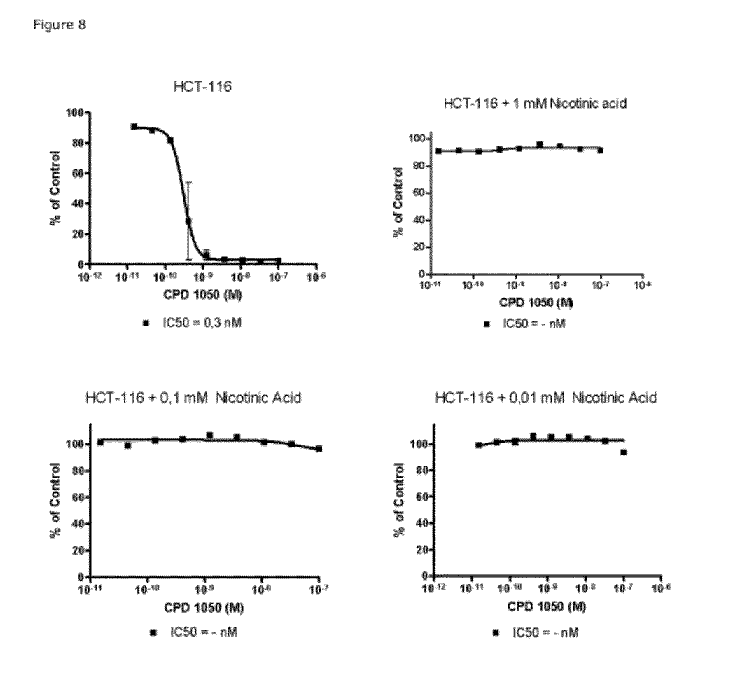

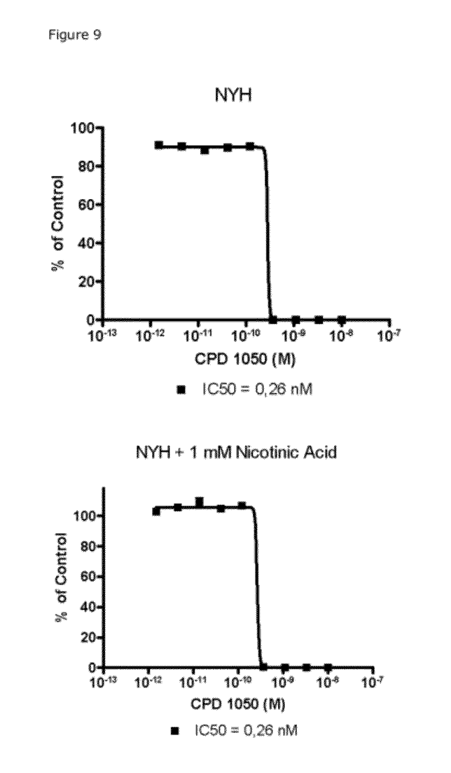

We investigated the in vitro rescue effects of nicotinic acid on tumour cells treated with a variety of NAMPRT inhibitors, to demonstrate that these findings are generally applicable to NAMPRTi and are not specific to APO866. The results shown in FIG. 7 show that the ovarian cancer cell line A2780 is minimally rescued by nicotinic acid from the cytotoxicity of the NAMPRT inhibitor CHS828. This is in agreement with the results for A2780 treated with APO866 as summarized in Table 1. The results in FIGS. 8 and 9 show that nicotinic acid can rescue HCT116 colon cancer cells from treatment with The NAMPRT inhibitor compound 1050, but NYH small cell lung cancer cells are not similarly protected. Again, this is in accord with the results for APO866 in these cell lines as shown in Table 1.

Table 1 illustrates the in vitro protection from APO866 by nicotinic acid. Rescue effect defined as >39-fold increase of IC₅₀ to APO866 treatment. No rescue effect defined as <2-fold increase in IC₅₀. NA=nicotinic acid.

| Cell line | NA rescue effect |
|---|---|
| ML-2 | + |
| HCT-116 | + |
| A431 | + |
| NYH | − |
| A2780 | − |

NAPRT Expression is a Marker for Nicotinic Acid Rescue in Cancer Cells

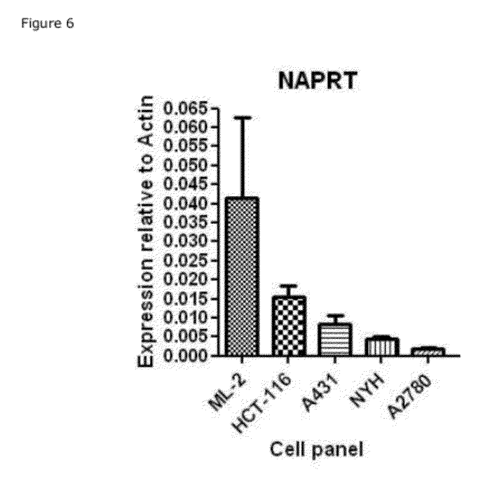

The rationale for nicotinic acid rescue from APO866 cytotoxicity is the utilization of the alternative NAD synthesis pathway with nicotinic acid as a substrate. The initial step in this pathway is catalyzed by the enzyme nicotinic acid phosphorribosyltransferase (NAPRT). We examined the mRNA expression of NAPRT in the panel of cancer cell lines. We find that the expression of NAPRT is highest in cell lines rescued with nicotinic acid and lowest in cell lines not protected from APO866 (FIG. 6). The expression in ML-2 cells is 24 fold higher than what is found in A2780 cells.

Cells not Utilizing Nicotinic Acid do not Express NAPRT at the Protein Level.

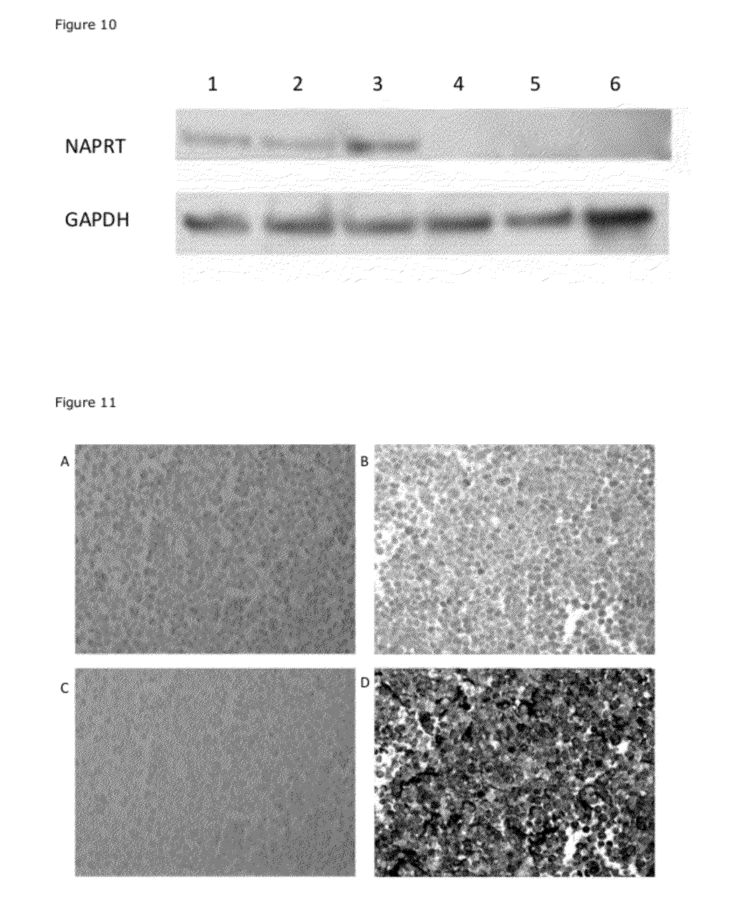

Having identified cell lines with and without the ability to utilize nicotinic acid for protection against NAMPRT inhibitors we studied the protein levels of NAPRT, the rate-limiting step in synthesis of NAD from nicotinic acid. Notably, we have found that for PC-3 cells the LD₅₀ values for APO866 are unaffected by nicotinic acid (4.8±3.0 nM without nicotinic acid (100 μM) present and 5.5±1.4 nM with). We found that in cell lines protected by nicotinic acid (ML-2, HCT-116 and A431) NAPRT was expressed whereas in cells not protected by nicotinic acid (A2780, NYH and PC-3) no detectable NAPRT protein was present (FIG. 10). Further, we investigated whether this difference could be observed by immunohistochemistry. We found that no positive reactivity was found for NAPRT in PC-3 (FIG. 11 A+C) cells while in HCT-116 cells (FIG. 11 B+D) a strong reactivity was observed. Thus, cells protected and unprotected against NAMPRT inhibitors by nicotinic acid can be clearly differentiated by expression levels of NAPRT using western blotting and immunohistochemistry.

The cytotoxic effect of APO866 on tumour cells is due to reduction of cellular NAD levels. We examined whether activating an alternative NAD synthesis pathway by supplementation of nicotinic acid could protect against adverse reactions and death in vivo in mice. Nicotinic acid protects against death even at four times the normal MTD of APO866 in mice if administered on the same days as APO866. Also the main marker for adverse reaction, thrombocytopenia, is ameliorated. In this respect, nicotinic acid can be used as an antidote for APO866 toxicity caused by accidental over-administration. This has previously also been found for the NAMPRT inhibitor GMX1777 indicating the protective effect of nicotinic acid to be effective against NAMPRT inhibitors in general (Beauparlant et al. Anti-cancer drugs 20[5]: 346-354.) Also, these results indicate that the dose-limiting toxicity of APO866 is target specific. This may make APO866 suitable for combination treatments.

When the mechanism of action of APO866 was discovered, the lack of protective effect of nicotinic acid by HEPG2 cells was perceived as a surprising but isolated case. However, we surprisingly find that in a broad panel of cancer cell lines the ability to utilize nicotinic acid to synthesize NAD to protect against NAMPRT inhibitors is only found in 60% of the cell lines. This also opens the possibility of exploiting the fact that when co-treating with nicotinic acid APO866 can be used at doses four times higher than normal MTD. We show a dramatic increase in % ILS in an A2780 xenograft mouse model when co-treating with nicotinic acid and high doses of APO866. This indicates an opportunity for better treatment in tumours unable to utilize nicotinic acid. It should be noticed that at lower concentrations of APO866 nicotinic acid displays some protection even though A2870 cells are unable to use it. This may be due to local or systemic conversion of nicotinic acid to nicotinamide and nicotinamide mononucleotide, increasing the circulating concentrations of these metabolites sufficiently to interfere with the APO866 treatment. We also show that in xenografts of ML-2, the co-treatment with nicotinic acid completely abolishes the anti-proliferative effects of APO866. This is seen even at high concentrations of APO866. This emphasizes the need for a marker for protection by nicotinic acid. Logically, the ability of cells to utilize nicotinic acid could be due to the expression levels of enzymes involved in the synthesis of NAD. NAPRT is the first step of NAD synthesis from nicotinic acid and the enzyme is not inhibited by APO866. We found the expression of NAPRT on the mRNA level to correlate with protection by nicotinic acid. We propose the expression of NAPRT as a marker for identifying cancers suitable for combination treatment with high dose APO866 and nicotinic acid. This could be from detection of NAPRT mRNA expression in tumour tissue or biopsies. Furthermore protein levels can be detected by immunohistochemistry, ELISA or other antibody based detection methods as an alternative way to identify tumours not utilizing nicotinic acid. Together, the increased dose tolerance of APO866 with nicotinic acid, and the possibility of identifying tumours not protected from APO866 and other NAMPRT inhibitors by nicotinic acid may increase the potential for NAMPRT inhibitor treatment in stratified subgroups of cancer patients.

1. A method for the treatment or for alleviating the symptoms of a cancer in a subject, the method comprising the steps of
a) determining the level of Nicotinic acid phosphoribosyltransferase (NAPRT) in said subject; and
b) 1) in the event of a level of NAPRT, as determined in step a) above, which is lower than a predetermined threshold value, treating said subject sequentially or simultaneous with i) an effective amount of a nicotinamide phosphoribosyltransferase inhibitor (NAMPRTi), and ii) an effective amount of a nicotinic acid, a nicotinic acid precursor or a prodrug of nicotinic acid; or
2) in the event of a level of NAPRT, as determined in step a) above, which is higher than or equal to a predetermined threshold value, treating said subject with i) an effective amount of a nicotinamide phosphoribosyltransferase inhibitor (NAMPRTi) in the absence of sequential or simultaneous treatment with ii) an effective amount of a nicotinic acid, a nicotinic acid precursor or a prodrug of nicotinic acid.

2. The method according to claim 1, wherein the sequential or simultaneous treatment with i) an effective amount of a nicotinamide phosphoribosyltransferase inhibitor (NAMPRTi), and ii) an effective amount of a nicotinic acid, a nicotinic acid precursor or a prodrug of nicotinic acid under step b)1) is sequential and within 24 hours of treatment.

3. The method according to claim 1, wherein the absence of sequential or simultaneous treatment with an effective amount of a nicotinic acid, a nicotinic acid precursor or a prodrug of nicotinic acid under step b)2) is sequential and within 24 hours of treatment.

4. The method according to claim 1, wherein said subject is treated subsequent and after 24 hours of the treatment under step b) with an effective amount of a nicotinic acid, a nicotinic acid precursor or a prodrug of nicotinic acid.

5. The method according to claim 1, wherein said level of NAPRT is determined in the tumour tissue of said subject.

6. The method according to claim 1, wherein said level of nicotinic acid phosphoribosyltransferase (NAPRT) is determined on the level of nucleic acids encoding NAPRT, such as by RT-PCR.

7. The method according to claim 1, wherein said level of Nicotinic acid phosphoribosyltransferase (NAPRT) is determined on the level of proteins, such as in assays based on specific antibodies or other specific binding partners to NAPRT.

8. The method according to claim 1, wherein said nicotinic acid, nicotinic acid precursor or prodrug of nicotinic acid is nicotinic acid.

9. The method according to claim 1, wherein said nicotinamide phosphoribosyltransferase inhibitor (NAMPRTi) is selected from the list consisting of:

| Compound | Structure |
|---|---|
| APO866/FK866 | 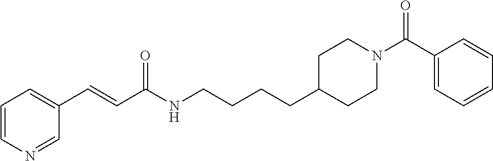 |
| CHS828/GMX1778 | 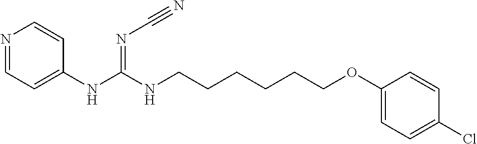 |
| EB1627/GMX1777 | 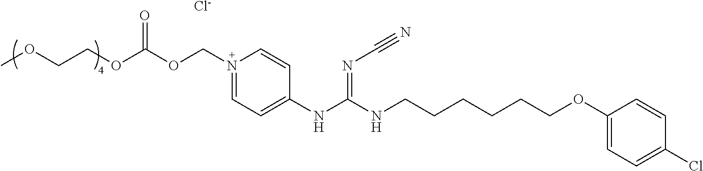 |
| Compound 1050 | 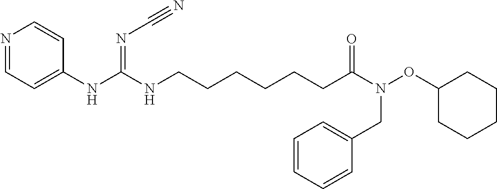 |
| Compound 1077 | 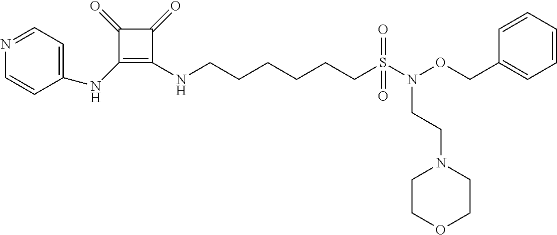 |
| Compound 1082 | 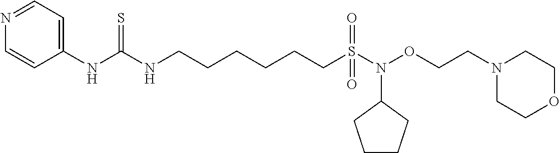 |
| Compound 1001 | 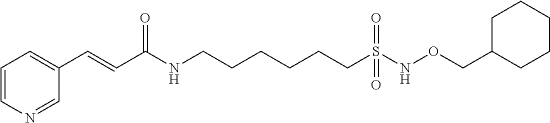 |
| Compound 1010 | 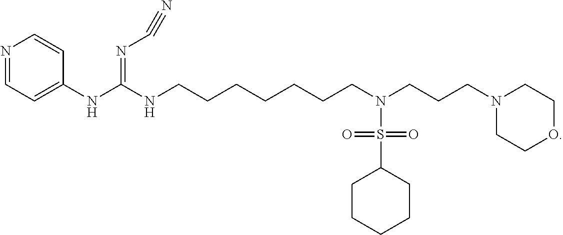 |

10. The method according to claim 1, wherein the effective amount of said nicotinic acid, a nicotinic acid precursor or a prodrug of nicotinic acid is administered intravenously at a dose of about 1 mg/day to about 3,000 mg/day.

11. The method according to claim 1, wherein the effective amount of said nicotinic acid, nicotinic acid precursor or prodrug of nicotinic acid is administered orally.

12. The method according to claim 1, wherein said nicotinamide phosphoribosyltransferase inhibitor (NAMPRTi) is administered prior to said nicotinic acid, nicotinic acid precursor or prodrug of nicotinic acid.

13. The method according to claim 1, wherein said nicotinamide phosphoribosyltransferase inhibitor (NAMPRTi) is administered concurrently with the administration of said nicotinic acid, nicotinic acid precursor or prodrug of nicotinic acid.

14. The method according to claim 1, which method further comprises administering said subject an effective amount of a DNA damaging agent.

15. A method of using Nicotinic acid phosphoribosyltransferase (NAPRT) as a biomarker in selecting responsive patients to the sequential or simultaneous treatment with i) an effective amount of a nicotinamide phosphoribosyltransferase inhibitor (NAMPRTi), and ii) an effective amount of a nicotinic acid, a nicotinic acid precursor or a prodrug of nicotinic acid.

16. A method of using Nicotinic acid phosphoribosyltransferase (NAPRT) as a biomarker in selecting patients that benefit from being treated with an effective amount of a nicotinamide phosphoribosyltransferase inhibitor (NAMPRTi) in the absence of sequential or simultaneous treatment with an effective amount of a nicotinic acid, a nicotinic acid precursor or a prodrug of nicotinic acid.

17. (canceled)

18. A method for alleviating the side effects of a nicotinamide phosphoribosyltransferase inhibitor (NAMPRTi) in the treatment with an effective amount of said NAMPRTi of a cancer in a subject, the method comprising the steps of
a) determining the level of Nicotinic acid phosphoribosyltransferase (NAPRT) in said subject; and
b) in the event of a level of NAPRT, as determined in step a) above, which is lower than a predetermined threshold value, treating said subject with an effective amount of a nicotinic acid, a nicotinic acid precursor or a prodrug of nicotinic acid, sequentially or simultaneous with the treatment with said effective amount of a nicotinamide phosphoribosyltransferase inhibitor (NAMPRTi).

19. The method according to claim 18, wherein the side effects is in normal tissue, such as lymphocytes and primary intestinal cells.
